SEQUENCE LISTING (8 sequences)

SEQ ID NO: 1 - DNA, 20 bp, Severe acute respiratory syndrome-related coronavirus
  source 1..20: mol_type = other DNA
acaccaaaagatcacattgg

SEQ ID NO: 2 - DNA, 20 bp, Severe acute respiratory syndrome-related coronavirus
  source 1..20: mol_type = other DNA
cccgcaatcctgctaacaat

SEQ ID NO: 3 - DNA, 22 bp, Alphainfluenzavirus influenzae
  source 1..22: mol_type = other DNA
ctagtactgtgtctacagtgtc

SEQ ID NO: 4 - DNA, 20 bp, Alphainfluenzavirus influenzae
  source 1..20: mol_type = other DNA
acaggaagcaaagcacaggg

SEQ ID NO: 5 - DNA, 13 bp, Alphainfluenzavirus influenzae
  source 1..13: mol_type = other DNA
tctacagtgtcaa

SEQ ID NO: 6 - DNA, 11 bp, Alphainfluenzavirus influenzae
  source 1..11: mol_type = other DNA
ctagtactgtg

SEQ ID NO: 7 - DNA, 19 bp, Betainfluenzavirus influenzae
  source 1..19: mol_type = other DNA
cggtggattaaacaaaagc

SEQ ID NO: 8 - DNA, 20 bp, Betainfluenzavirus influenzae
  source 1..20: mol_type = other DNA
gccaatggaaccaaatatag

(19) United States
(12) Patent Application Publication
(10) Pub. No.: US 2025/0003965 A1
(43) Pub. Date: Jan. 2, 2025

(54) NUCLEIC ACID-BASED MULTIPLEXED LATERAL FLOW ASSAY FOR DISEASE DETECTION

(71) Applicants: The Penn State Research Foundation, University Park, PA (US); University of Maryland, Baltimore, Baltimore, MD (US); University of Maryland, Baltimore County, Baltimore, MD (US)

(72) Inventors: Dipanjan Pan, Ellicott City, MD (US); Parikshit Moitra, Baltimore, MD (US); Maha Mohammad Shehadeh Alafeef, Baltimore, MD (US); Ketan Dighe, Ellicott City, MD (US)

(21) Appl. No.: 18/756,459

(22) Filed: Jun. 27, 2024

Related U.S. Application Data
(60) Provisional application No. 63/524,025, filed on Jun. 29, 2023.

(51) Int. Cl. G01N 33/543; C12Q 1/70; G01N 21/77; G01N 21/78

(52) U.S. Cl. CPC G01N 33/54388; C12Q 1/701; G01N 21/78; G01N 33/54346; G01N 2021/7759; G01N 2333/11; G01N 2333/165

(57) ABSTRACT

Embodiments relate to compositions, methods, and systems for screening and detecting respiratory diseases. In particular, embodiments relate to a lateral flow assay configured to simultaneously detect the presence of one or more target gene sequences of respiratory diseases of interest. Embodiments may utilize antisense oligonucleotides designed specifically to bind in complementary fashion to a target gene sequence of a respiratory disease of interest

### CROSS-REFERENCE TO RELATED APPLICATIONS

This patent application is related to and claims the benefit of priority of U.S. Provisional Application 63/524,025, filed on Jun. 29, 2023, the entire contents of which is incorporated by reference.

### STATEMENT REGARDING FEDERALLY SPONSORED RESEARCH DEVELOPMENT

This invention was made with government support under Grant No. EB028026 awarded by the National Institutes of Health. The Government has certain rights in the invention.

### INCORPORATION BY REFERENCE STATEMENT REGARDING SEQUENCE LISTINGS

A Sequence Listing using extensible Markup Language (XML) compliant with World Intellectual Property Organization (WIPO) Standard ST.26 is provided herewith and the entirety of this sequence listing is incorporated by reference herein. The Sequence Listing that is incorporated by reference herein is e-filed at the USPTO using Patent Center as an XML file, named 0073605-000863.xml (8 KB in size, created on Jun. 25, 2024).

### FIELD

Embodiments relate to compositions, methods, and systems for screening and detecting respiratory diseases. In particular, embodiments relate to amplification-free lateral flow assays configured to simultaneously detect the presence of one or more target gene sequences of respiratory diseases of interest.

### BACKGROUND

The coronavirus disease 2019 (COVID-19) was caused by a novel severe acute respiratory syndrome-coronavirus-2 (SARS-COV-2). SARS-COV-2 and influenza virus (Flu A/B) are major pathogens that primarily target the human respiratory system. SARS-COV-2 causes nonspecific symptoms, and the onset of this disease coincides with the active Flu A/B season. The non-specific symptoms and scarcity of clinical knowledge of the SARS-COV-2 infection can mislead patient diagnoses. Considering the difficulty in diagnosing simultaneous infection of SARS-COV-2 and Flu A/B, it is difficult to subjectively analyze the overlapping clinical symptoms or frequency of these two viral infections. Approximately 3% of respiratory infections occur simultaneously with SARS-COV-2 infection, followed by Flu A/B. Co-infection may also lead to more severe symptoms than infection caused by a single virus. A crucial shortcoming of healthcare systems across the globe has been the ability to rapidly and accurately detect co-infection, with contributing factors such as shortages of test kits and specimen materials, and no established funding mechanism to support these testing facilities. Furthermore, current gold standard tests (i.e., RT-PCR) for diagnosing infection due to SARS-COV-2 or Flu A/B are complex and labor-intensive, requiring each sample to be sent to a laboratory for confirmation.

### SUMMARY

Several immunodiagnostic and serological assays have been developed to detect either the presence of antibodies or antigens against SARS-COV-2 and Flu A/B. However, the utility of most of these techniques is limited. While immunological tests suffer from a detectable antibody response at the early stages of infection, serological tests have drawbacks such as cross-reactivity with other pathogens, including other human coronaviruses. Thus, both these approaches may contribute to high incidence of either false positive or false negative results, respectively.

Nucleic acid-based assays (NAA) and nucleic acid based amplification tests (NAAT) offer the advantage of being highly specific and selective for a particular pathogen. For NAAT, the detection principle majorly encounters the complex requirement of amplification of cDNA extracted from the virus, e.g., SARS-COV-2 RNA collected from bio-fluids of subjects. Based on this principle, numerous RT-PCR kits have been developed for diagnosis of COVID-19. However, current RT-PCR tests for diagnosing COVID-19 and Flu A/B are complex and labor-intensive. Furthermore, technologies such as the Cepheid Xpert® Xpress SARS-COV-2 test can provide results for the detection of SARS-COV-2 in approximately 45 minutes with low false-negative rate at 1.8% as demonstrated in an independent study. However, this test requires the GeneXpert system, of which there are only 5,000 systems available in the US. The test also requires RNA extraction as a separate step from amplification and detection, which is a key constraint on scalability and could become important as demand increases for critical supplies. The Abbott ID NOW™ COVIS-19 test uses isothermal amplification technology and claims the delivery of positive results in less than 15 minutes while offering a device with portable size and weight. However, this test also requires a specialized instrument and availability issues as well as accuracy issues have been recently reported for this test.

We determined that there is an ongoing need for a system configured to detect co-infection and differentiate between SARS-COV-2 and Flu A/B. As many existing techniques remain laborious and technically challenging, there is a need for a rapid, cost-effective, and selective diagnostic test for SARS-COV-2 that can provide fast and accurate test results quickly to effectively mitigate the spread of SARS-COV-2 and differentiate it from Flu A/B. A system that can be deployed for self-testing at home and that provides rapid results with the flexibility to differentiate between SARS-COV-2 and Flu A/B will be an important diagnostic advancement to reduce the public burden of disease transmission.

Therefore, we have developed a series of antisense oligonucleotides (ASOs) targeting different genetic segments of respiratory diseases, such as SARS-COV-2 and Flu A/B, for use in a lateral flow assay. In particular, the lateral flow assay may utilize functionalized ASOs that allow for differentiation between positive and negative samples within a short timeframe after addition of a sample to a testing strip. The designed ASOs offer several advantages over other tests currently used for disease detection, including user-friendliness, affordability, portability POC diagnostics, and ability to be used in regions with a lack of laboratory-grade infrastructure and resources.

In an exemplary embodiment, an apparatus for detecting one or more respiratory diseases in a sample comprises first antisense oligonucleotides functionalized with a first small molecule at their first ends, wherein the first antisense oligonucleotides have a sequence that is complementary of a first target gene sequence of a first respiratory disease; second antisense oligonucleotides functionalized with a second small molecule at their second ends, wherein the second antisense oligonucleotides have a sequence that is complementary of a second target gene sequence of the first respiratory disease; third antisense oligonucleotides functionalized with a third small molecule at their first ends, wherein the third small molecule is the same as the first small molecule, wherein the third antisense oligonucleotides have a sequence that is complementary of a first target gene sequence of a second respiratory disease; and fourth antisense oligonucleotides functionalized with a fourth small molecule at their second ends, wherein the fourth small molecule is different than the second small molecule, wherein the fourth antisense oligonucleotides have a sequence that is complementary of a second target gene sequence of the second respiratory disease.

In some embodiments, the first and second respiratory diseases are two different diseases selected from the group consisting of SARS-COV-2, Flu A, and Flu B.

In some embodiments, the apparatus further comprises fifth antisense oligonucleotides functionalized with a fifth small molecule at their first ends, wherein the fifth small molecule is the same as the first and third small molecules, wherein the fifth antisense oligonucleotides have a sequence that is complementary of a first target gene sequence of a third respiratory disease; and sixth antisense oligonucleotides functionalized with a sixth small molecule at their second ends, wherein the sixth small molecule is different than the second and fourth small molecules, wherein the sixth antisense oligonucleotides have a sequence that is complementary of a second target gene sequence of the third respiratory disease.

In some embodiments, the first, second, and third respiratory diseases are three different diseases selected from the group consisting of SARS-COV-2, Flu A, and Flu B.

In some embodiments, the first or second respiratory disease is SARS-COV-2, the first and second target gene sequences of SARS-COV-2 are SEQ ID NO 1 (ACACCAAAAGATCACATTGG) and SEQ ID NO 2 (CCCGCAATCCTGCTAACAAT), when the first or second respiratory disease is Flu A, the first and second target gene sequences of Flu A are selected from SEQ ID NO 3 (CTAGTACTGTGTCTACAGTGTC) and SEQ ID NO 4 (ACAGGAAGCAAAGCACAGGG), and SEQ ID NO 5 (TCTACAGTGTCAA) and SEQ ID NO 6 (CTAGTACTGTG), and when the first or second respiratory disease is Flu B, the first and second target gene sequences of Flu B are SEQ ID NO 7 (CGGTGGATTAAACAAAAGC) and SEQ ID NO 8 (GCCAATGGAACCAAATATAG).

In some embodiments, the first small molecule and the third small molecule is a small molecule selected from the group consisting of biotin, 6-carboxyfluorescein (6-FAM), fluorescein isothiocyanate (FITC), and digoxigenin (DIG).

In some embodiments, the second small molecule and fourth small molecule are two different small molecules selected from the group consisting of biotin, 6-carboxyfluorescein (6-FAM), fluorescein isothiocyanate (FITC), and digoxigenin (DIG).

In some embodiments, the apparatus further comprises a testing strip comprising a sample application region, a control region, a first testing region configured to detect the presence of the first respiratory disease, and a second testing region configured to detect the presence of the second respiratory disease, wherein the first and second testing regions are positioned between the sample application region and the control region, and wherein the sample application region is configured to receive the sample, which is configured flow through the testing strip towards the first and second testing regions and the control region.

In some embodiments, the first testing region has first capture compounds immobilized on the testing strip, wherein the first capture compounds are configured to capture the second small molecule of the second antisense oligonucleotides.

In some embodiments, the second testing region has second capture compounds immobilized on the testing strip, wherein the second capture compounds are configured to capture the fourth small molecule of the fourth antisense oligonucleotides.

In some embodiments, the testing strip has nanoparticles configured to flow through the testing strip, wherein some of the nanoparticles are configured to capture the first small molecule of the second antisense oligonucleotides, and wherein some of the nanoparticles are configured to capture the third small molecule of the fourth antisense oligonucleotides.

In some embodiments, the nanoparticles are configured to effectuate a color change when reacted and immobilized on the testing strip.

In some embodiments, the apparatus further comprises positively charged molecules configured to flow through the testing strip, wherein the positively charged molecules are configured to bind to the nanoparticles and augment the color change effectuated by the nanoparticles.

In some embodiments, the control region has third capture compounds immobilized on the testing strip, wherein the third capture compounds are configured to capture some of the nanoparticles.

In an exemplary embodiment, a method for detecting one or more respiratory diseases in a sample comprises providing a sample solution. The sample solution comprises a collected sample including nucleic acid from a subject, first antisense oligonucleotides functionalized with a first small molecule at their first ends, wherein the first antisense oligonucleotides have a sequence that is complementary of a first target gene sequence of a first respiratory disease, second antisense oligonucleotides functionalized with a second small molecule at their second ends, wherein the second antisense oligonucleotides have a sequence that is complementary of a second target gene sequence of the first respiratory disease, third antisense oligonucleotides functionalized with a third small molecule at their first ends, wherein the third small molecule is the same as the first small molecule, wherein the third antisense oligonucleotides have a sequence that is complementary of a first target gene sequence of a second respiratory disease, and fourth antisense oligonucleotides functionalized with a fourth small molecule at their second ends, wherein the fourth small molecule is different than the second small molecule, wherein the fourth antisense oligonucleotides have a sequence that is complementary of a second target gene sequence of the second respiratory disease. The method further comprises providing a testing strip comprising a sample application region, a control region, a first testing region configured to detect the presence of the first respiratory disease, and a second testing region configured to detect the presence of the second respiratory disease, wherein the first and second testing regions are positioned between the sample application region and the control region; and applying the sample solution at the sample application region.

In some embodiments, the first and second respiratory diseases are two different diseases selected from the group consisting of SARS-COV-2, Flu A, and Flu B.

In some embodiments, the sample solution further comprises fifth antisense oligonucleotides functionalized with a fifth small molecule at their first ends, wherein the fifth small molecule is the same as the first and third small molecules, wherein the fifth antisense oligonucleotides have a sequence that is complementary of a first target gene sequence of a third respiratory disease; and sixth antisense oligonucleotides functionalized with a sixth small molecule at their second ends, wherein the sixth small molecule is different than the second and fourth small molecules, wherein the sixth antisense oligonucleotides have a sequence that is complementary of a second target gene sequence of the third respiratory disease.

In some embodiments, the first, second, and third respiratory diseases are three different diseases selected from the group consisting of SARS-COV-2, Flu A, and Flu B.

In some embodiments, when the first or second respiratory disease is SARS-COV-2, the first and second target gene sequences of SARS-COV-2 are SEQ ID NO 1 and SEQ ID NO 2, when the first or second respiratory disease is Flu A, the first and second target gene sequences of Flu A are selected from SEQ ID NO 3 and SEQ ID NO 4, and SEQ ID NO 5 and SEQ ID NO 6, and when the first or second respiratory disease is Flu B, the first and second target gene sequences of Flu B are SEQ ID NO 7 and SEQ ID NO 8.

In some embodiments, the first testing region has first capture compounds immobilized on the testing strip, wherein the first capture compounds are configured to capture the second small molecule of the second antisense oligonucleotides.

In some embodiments, the second testing region has second capture compounds immobilized on the testing strip, wherein the second capture compounds are configured to capture the fourth small molecule of the fourth antisense oligonucleotides.

In some embodiments, the testing strip has nanoparticles configured to flow through the testing strip, wherein some of the nanoparticles are configured to capture the first small molecule of the firth antisense oligonucleotides, and wherein some of the nanoparticles are configured to capture the third small molecule of the third antisense oligonucleotides.

In some embodiments, the nanoparticles are configured to effectuate a color change when reacted and immobilized on the testing strip.

In some embodiments, the method further comprises applying positively charged molecules configured to flow through the testing strip, wherein the positively charged molecules are configured to bind to the nanoparticles and augment the color change effectuated by the nanoparticles.

### BRIEF DESCRIPTION OF THE DRAWINGS

The above and other objects, aspects, features, advantages, and possible applications of embodiments of the present innovation will be more apparent from the following more particular description thereof, presented in conjunction with the following drawings. Like reference numbers used in the drawings may identify like components.

FIG. 1 is a schematic illustration of an exemplary lateral flow assay system for screening and detecting respiratory diseases. The presence of a first target gene sequence may be indicated by a prominent test (T1) line, the presence of a second target gene sequence may be indicated by a prominent test (T2) line, the presence of a third target gene sequence may be indicated by a prominent test (T3) line, along with a control (C) line.

FIG. 2 is a schematic illustration of an exemplary process for screening and detecting respiratory diseases using a lateral flow assay.

FIG. 3 is a schematic illustration of an exemplary workflow for screening and detecting respiratory diseases using a lateral flow assay.

FIG. 4 shows results from RNA isolated via NAP-10 column and direct lateral flow assay (left), and results before and after addition of cysteamine AuNPs (right).

FIG. 5 shows results from a sensitivity and selectivity study utilizing SARS-COV-2 genomic RNA to investigate the analytic detection limit of the system for SARS-COV-2. The top image shows changes in the test line at different concentrations of the SARS-COV-2 genomic RNA (a: 67250 copies/μL; b: 3362 copies/μL; c: 168 copies/μL; d: 8 copies/μL; e: 0.42 copies/μL; f: 0.02 copies/μL; g: 0.001 copies/μL). The bottom image shows cross-reactivity of the designed lateral flow assay system using different synthetic RNAs from various microorganisms (a: Zika; b: Influenza A; c: Influenza B; d: feline infectious peritonitis virus (FIPV); e: SARS-COV; f: MERS-COV and g: SARS-COV-2).

FIG. 6 shows lateral flow assays tested against SARS-COV-2 and Flu B, and SARS-COV-2-and Flu A.

### DETAILED DESCRIPTION

The following description is of exemplary embodiments and methods of use that are presently contemplated for carrying out the present invention. This description is not to be taken in a limiting sense but is made merely for the purpose of describing the general principles and features of various aspects of the present invention. The scope of the present invention is not limited by this description.

Embodiments generally relate to compositions, methods, and systems configured to accurately screen and detect respiratory diseases. Exemplary compositions may comprise antisense oligonucleotides (ASOs) configured to selectively detect a target gene sequence of a respiratory disease of interest.

### Lateral Flow Assay

Embodiments relate to a lateral flow assay system and method configured to receive and analyze a sample to determine if the sample comprises at least one target gene sequence related to at least one respiratory disease of interest. The lateral flow assay system and method can be used as a point-of-care (POC) test, for example as a rapid lab test, for screening and detection of respiratory diseases.

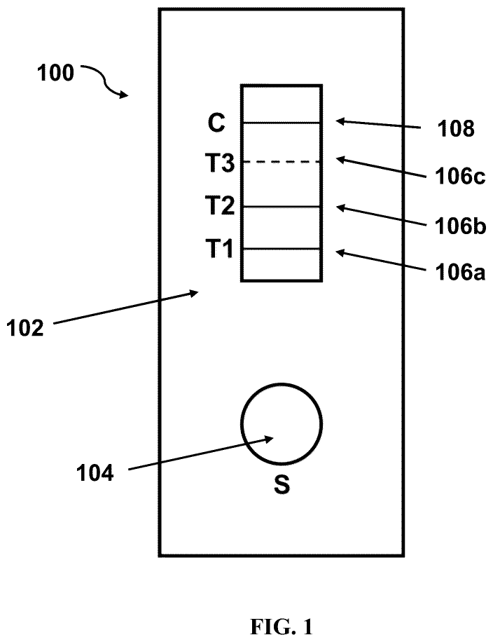

As seen in FIG. 1, a lateral flow system 100 may comprise a testing strip 102. The testing strip 102 may comprise a sample application region 104, at least one testing region (e.g., a first testing region 106a, a second testing region 106b, and a third testing region 106c), and a control region 108. It is contemplated that a sample collected from a subject, or a solution comprising a sample collected from a subject, may be placed on or at the sample application region 104 and flow through the testing strip 102 (e.g., across the length of the testing strip 102) thereafter. The sample application region 104 may therefore be designated as the beginning of the test strip 102. It is contemplated that the at least one testing region may be positioned in between the sample application region 104 and the control region 108 such that the sample may flow from the sample application region 104, then to the at least one testing region, then to the control region 108.

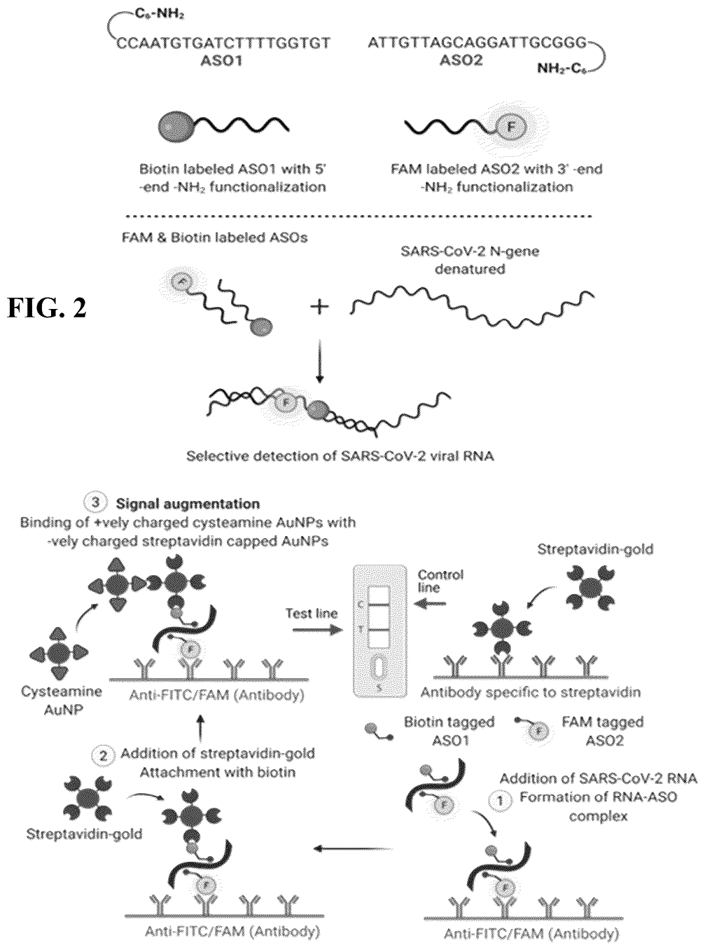

In exemplary embodiments, the lateral flow system 100 may further comprise ASOs configured to detect one or more respiratory diseases present in the sample (see FIG. 2). The ASOs are single-stranded DNA sequences designed specifically to bind in complementary fashion to a target gene sequence of a respiratory disease of interest. For example, ASOs have nucleotide sequences that complement the nucleotide sequence of a target gene (e.g., adenine (A) in an ASO sequence may complement and bind to thymine (T) in a target gene sequence, cytosine (C) in an ASO sequence may complement and bind to guanine (G) in a target gene sequence, thymine (T) in an ASO sequence may complement and bind to adenine (A) in a target gene sequence, and guanine (G) in an ASO sequence may complement and bind to cytosine (C) in a target gene sequence). The ASOs may alternatively be referred to as capture and detection probes herein.

The capture and detection probes may be functionalized with a moiety at either their first end or their second end such that their first end or their second end may bind to a small molecule. It is contemplated that the first end can be a five prime end (5′ end) and the second end can be a three prime end (3′ end). In some embodiments, the capture and detection probes are functionalized with an amine (—NH₂) moiety or a thiol (—SH) moiety at either their first end or their second end, and the amine moiety or thiol moiety may then be used to couple to a small molecule. As the amine and thiol moieties may couple to both the capture and detection probes and the small molecules, when the capture and detection probes bind to their target gene sequences, the small molecules are also necessarily present at the target gene sequences.

In some embodiments, capture and detection probes may be functionalized at their first ends with first small molecules. In preferred embodiments, the first small molecule may be biotin, 6-carboxyfluorescein (6-FAM), fluorescein isothiocyanate (FITC), digoxigenin (DIG), or any other suitable small molecule.

In some embodiments, capture and detection probes may alternatively be functionalized at their second ends with second small molecules. In preferred embodiments, the second small molecules may be biotin, 6-FAM, FITC, DIG, or any other suitable compound.

In exemplary embodiments, capture and detection probes may be chosen in pairs and may be configured to bind to two closely spaced regions of a target gene sequence. It is contemplated that each capture and detection probes of a pair may be differentially functionalized (e.g., the first capture and detection probe functionalized at its first end and the second capture and detection probe functionalized at its second end) such that the functionalized ends of each capture and detection probe are in close proximity to each other when the capture and detection probes are bound to their respective regions of the target gene sequence. For example, a first capture and detection probe functionalized at its first end may be complementary of a first region of a target gene sequence, and a second capture and detection probe functionalized at its second end may be complementary of a second region of a target gene sequence that is in close proximity to the first region. It is contemplated that capture and detection probe pairs may ensure that the screening and detection will not fail even if one region of a target gene sequence undergoes mutation.

It is contemplated that the capture and detection probes may be designed to target gene sequences that are less prone to mutation and/or antibiotic resistance. For example, capture and detection probes may be designed to target regions that may be conserved among different strains of a respiratory disease and less prone to antibiotic resistance. By targeting conserved regions, capture and detection probes may be used universally for diagnostic purposes, ensuring consistent and reliable results regardless of genetic variations among strains.

In some embodiments, the system may comprise multiple capture and detection probe pairs configured to complement and bind to multiple regions of a single target gene sequence. An advantage of using multiple capture and detection probe pairs is to ensure recognition of at least one region of the target gene sequence even if other regions of the target gene sequence undergo or are subject to mutations.

The system may comprise any number of capture and detection probe pairs. The system may comprise at least a first capture and detection probe pair configured to complement and bind to a first target gene sequence, a second capture and detection probe pair configured to complement and bind to a second target gene sequence, a third capture and detection probe pair configured to complement and bind to a third target gene sequence, etc. It is contemplated that the different target gene sequences may correspond to different gene sequences of the same respiratory disease. It is further contemplated that the different target gene sequences may correspond to gene sequences of different respiratory diseases. For example, the first target gene sequence may correspond to a first respiratory disease, the second target gene sequence may correspond to a second respiratory disease, the third target gene sequence may correspond to a third respiratory disease, the fourth target gene sequence may correspond to a fourth respiratory disease, etc. Accordingly, a single system may be used for screening and detection of various respiratory diseases.

It is contemplated that the system can be configured to detect one or more respiratory diseases, including but not limited to, SARS-COV-2, Flu A, and Flu B, though it is contemplated that the system can be configured to detect any respiratory disease. Accordingly, the system can include capture and detection probes configured to complement and bind to a target gene sequence corresponding to SARS-COV-2, and/or capture and detection probes configured to complement and bind to a target gene sequence corresponding to Flu A, and/or capture and detection probes configured to complement and bind to a target gene sequence corresponding to Flu B, and/or capture and detection probes configured to complement and bind to any other respiratory disease of interest.

In some embodiments, the system is configured to detect the presence of one or both of two predetermined respiratory diseases. For example, the system may be configured to detect one or both of SARS-COV-2 and Flu A, or one or both of SARS-COV-2 and Flu B. In some embodiments, the system is configured to detect the presence of one, both, or all of three predetermined respiratory diseases. For example, the system may be configured to detect one, both, or all of SARS-COV-2, Flu A, and Flu B. Examples of such systems can be configured so that the detection of multiple predetermined respiratory diseases can be detected via the same detection mechanism having different capture and detection probe pairs. For example, some embodiments can be configured to detect SARS-COV-2 and/or Flu A and/or Flu B and/or any other respiratory disease of interest via a single platform having multiple different capture and detection probe pairs.

It is contemplated that capture and detection probes configured to detect different respiratory diseases may have unique second small molecules. For example, capture and detection probes configured to detect a first respiratory disease may have a different second small molecule than the second small molecules of capture and detection probes configured to detect a second respiratory disease, ASOs configured to detect a third respiratory disease, etc.

It is further contemplated that capture and detection probes configured to detect different respiratory diseases may have the same first small molecules. For example, capture and detection probes configured to detect a first respiratory disease may have the same first small molecules as the first small molecules of capture and detection probes configured to detect a second respiratory disease, capture and detection probes configured to detect a third respiratory disease, etc.

Exemplary methods and systems for screening and detecting respiratory diseases may comprise collecting a sample (e.g., an RNA/DNA sample) from a subject. It is contemplated that the sample may be collected using any suitable means, including but not limited to, an oral swab, a nasal swab, a cervical swab, a blood collecting swab, urine collection, or any other suitable means for collecting nucleic acid from the subject. It is further contemplated that the sample may be collected using any suitable instrument, including but not limited to, a cotton swab or any other suitable instrument for collecting nucleic acid from the subject.

The collected sample may then be introduced to a sensing solution to form an aqueous mixture. The sensing solution may comprise a plurality of capture and detection probe pairs (e.g., targeting a plurality of different gene sequences, as described above). For example, the sensing solution may comprise a first capture and detection probe pair configured to target a first target gene sequence of a first respiratory disease, a second capture and detection probe pair configured to target a second target gene sequence of a second respiratory disease, a third capture and detection probe pair configured to target a third target gene sequence of a third respiratory disease, etc. It is contemplated that the first capture and detection probe pair, the second capture and detection probe pair, the third capture and detection probe pair, etc. may have the same first small molecules but have unique/different second small molecules.

In some embodiments, the sensing solution may comprise at least a first capture and detection probe pair configured to target a first target gene sequence of SARS-COV-2 and at least a second capture and detection probe pair configured to target a second target gene sequence of Flu A or Flu B. It is contemplated that when the collected sample comprises a first target gene sequence (e.g., corresponding to SARS-COV-2), the first capture and detection probe pair may bind to the complementary first target gene sequence. It is further contemplated that when the collected sample comprises a second target gene sequence of (e.g., corresponding to Flu A or Flu B), the second capture and detection probe pair may bind to the complementary second target gene sequence. However, when the collected sample does not comprise any of the target gene sequences, the capture and detection probes may not bind to the nucleic acid of the sample.

In other embodiments, the sensing solution may comprise at least a first capture and detection probe pair configured to target a first target gene sequence of SARS-COV-2 at least a second capture and detection probe pair configured to target a second target gene sequence of Flu A, and at least a third capture and detection probe pair configured to target a third target gene sequence of Flu B. It is contemplated that when the collected sample comprises a first target gene sequence (e.g., corresponding to SARS-COV-2), the first capture and detection probe pair may bind to the complementary first target gene sequence. It is further contemplated that when the collected sample comprises a second target gene sequence of (e.g., corresponding to Flu A), the second capture and detection probe pair may bind to the complementary second target gene sequence. It is further contemplated that when the collected sample comprises a third target gene sequence of (e.g., corresponding to Flu B), the second capture and detection probe pair may bind to the complementary third target gene sequence. However, when the collected sample does not comprise any of the target gene sequences, the capture and detection probes may not bind to the nucleic acid of the sample.

In some embodiments, the sensing solution may further comprise a nucleic acid extraction buffer configured to extract nucleic acids from the collected sample. In alternative embodiments, nucleic acids may not be extracted from the collected sample prior to detection of a target gene sequence. Extraction of nucleic acid and amplification of nucleic acid may be performed but are not requirements for using the lateral flow system, thus allowing sensing of a target gene sequence directly from the collected sample.

In some embodiments, the aqueous mixture may be incubated prior to application to the testing strip 102. Incubation may include maintaining the aqueous mixture for a period of time under predetermined conditions. For example, the aqueous mixture may be incubated for at least 5 minutes at or near room temperature.

After formation of the aqueous mixture, the aqueous mixture may be placed at or near the sample application region 104. The aqueous mixture may then flow via capillary action through the test strip 102 in a flow direction and towards the testing regions 106a, 106b and control region 108. The speed of the flow may be affected by a number of factors, such as the types, quality and size of the flow strip used.

In exemplary embodiments, the system may further comprise an augmentation solution configured to be placed at or near the sample application region 104 with or after application of the aqueous mixture. The augmentation solution includes positively charged molecules configured to augment a signal produced by the capture and detection probes such that test results may be more clearly seen by a user. The positively charged molecules may be cationic nanoparticles selected from the group consisting of cysteine capped plasmonic (e.g., gold) nanoparticles, cysteamine capped plasmonic (e.g., gold) nanoparticles, and tris (2-aminoethyl) amine capped plasmonic (e.g., gold) nanoparticles, or may be cationic small molecules (e.g., methylene blue). The augmentation solution and positively charged molecules are described in further detail below.

The first testing region 106a may correspond to a first respiratory disease of interest and may comprise first capture compounds, such as first capture antibodies. The first capture compounds may be immobilized at the first testing region 106a such that the first capture compounds may not flow with the aqueous mixture as it flows through the testing strip 102. The first capture compounds are configured to capture the second small molecules of the first capture and detection probe pair corresponding to a first respiratory disease of interest. Accordingly, in embodiments wherein the mixture has target gene sequences corresponding to the first respiratory disease of interest and capture and detection probes coupled to the target gene sequences, the first capture compounds may bind to the capture and detection probes, thereby immobilizing the capture and detection probes at the first testing region 106a.

The second testing region 106b may correspond to a second respiratory disease of interest and may comprise second capture compounds, such as second capture antibodies. The second capture compounds may be immobilized at the second testing region 106b such that the second capture compounds may not flow with the aqueous mixture as it flows through the testing strip 102. The second capture compounds are configured to capture the second small molecules of the second capture and detection probe pair corresponding to a second respiratory disease of interest. Accordingly, in embodiments wherein the aqueous mixture has target gene sequences corresponding to the second respiratory disease of interest and capture and detection probes coupled to the target gene sequences, the second capture compounds may bind to the capture and detection probes, thereby immobilizing the capture and detection probes at the second testing region 106b.

The third testing region 106c may correspond to a third respiratory disease of interest and may comprise third capture compounds, such as third capture antibodies. The third capture compounds may be immobilized at the third testing region 106c such that the third capture compounds may not flow with the aqueous mixture as it flows through the testing strip 102. The third capture compounds are configured to capture the second small molecules of a third ASO pair corresponding to a third respiratory disease of interest. Accordingly, in embodiments wherein the aqueous mixture has target gene sequences corresponding to the third respiratory disease of interest and capture and detection probes coupled to the target gene sequences, the third capture compounds may bind to the capture and detection probes, thereby immobilizing the capture and detection probes at the third testing region 106c.

As capture and detection probe pairs may have unique/different second small molecules and the testing regions may have different capture compounds corresponding to different second small molecules, the testing regions may accurately correspond to different respiratory diseases. For example, in embodiments with two testing regions, the first capture and detection probe pair and the second capture and detection probe pair have unique/different second small molecules and the first and second testing regions 106a, 106b have different capture compounds corresponding to the different second small molecules, such that the first and second testing regions 106a, 106b may accurately correspond to different respiratory diseases. As another example, in embodiments with three testing regions, the first capture and detection probe pair, the second capture and detection probe pair, and the third capture and detection probe pairs have unique/different second small molecules and the first, second, and third testing regions 106a, 106b, 106c have different capture compounds corresponding to the different second small molecules, such that the first, second, and third testing regions 106a, 106b, 106c may accurately correspond to different respiratory diseases.

The testing strip 102 may further comprise nanoparticles temporarily immobilized on the testing strip 102. The nanoparticles may only mobilize/flow as the aqueous mixture flows through the testing strip 102. For example, the nanoparticles may be dehydrated on the testing strip 102 and may only mobilize/flow after being rehydrated by the aqueous mixture.

The nanoparticles may be configured to couple to the first small molecules of the capture and detection probe pairs. As the capture and detection probes may be immobilized by the first capture compounds at the first testing region 106a and/or by the second capture compounds at the second testing region 106b and/or by the third capture compounds at the third testing region 106c, it is contemplated that at least some of the nanoparticles may also be immobilized at the first testing region 106a and/or the second testing region 106b and/or the third testing region 106c as the nanoparticles flow through the test strip. It is further contemplated that at least some of the nanoparticles may not bind to the capture and detection probes and flow with the aqueous mixture as it flows through the testing strip 102 past the testing regions 106a, 106b, and/or 106c (e.g., towards the control region 108). In embodiments wherein the aqueous mixture does not have target gene sequences, the nanoparticles may flow freely with the aqueous mixture as it flows through the testing strip 102 past the testing regions 106a, 106b, and/or 106c (e.g., towards the control region 108).

The nanoparticles may further be configured to effectuate a color change when reacted and immobilized at a region. In some embodiments, when the nanoparticles are immobilized at the first testing region 106a due to the presence of a first target gene sequence in the sample, the nanoparticles may effectuate a color change at the first testing region 106a. In some embodiments, the color change may appear as a visible line or mark at the first testing region 106a. It is therefore contemplated that the presence of a line or mark at the first testing region 106a signals the presence of the first respiratory disease of interest. However, no line or mark at the first testing region 106a signals the absence of the first respiratory disease of interest in the sample. Similarly, when the nanoparticles are immobilized at the second testing region 106b due to the presence of a second target gene sequence in the sample, the nanoparticles may effectuate a color change at the second testing region 106b. In some embodiments, the color change may appear as a visible line or mark at the second testing region 106b. It is therefore contemplated that the presence of a line or mark at the second testing region 106b signals the presence of the second respiratory disease of interest. However, no line or mark at the second testing region 106b signals the absence of the second respiratory disease of interest in the sample. Additionally, when the nanoparticles are immobilized at the third testing region 106c due to the presence of a third target gene sequence in the sample, the nanoparticles may effectuate a color change at the third testing region 106c. In some embodiments, the color change may appear as a visible line or mark at the third testing region 106c. It is therefore contemplated that the presence of a line or mark at the third testing region 106c signals the presence of the third respiratory disease of interest. However, no line or mark at the third testing region 106c signals the absence of the third respiratory disease of interest in the sample.

In preferred embodiments, the nanoparticles are streptavidin capped plasmonic (e.g., gold) nanoparticles configured to couple to a small molecules, such as biotin, or any other suitable nanoparticles.

As described above, the system 100 may further comprise an augmentation solution comprising positively charged molecules. The positively charged molecules may flow through the testing strip 102 in a similar manner as the sensing solution. In particular, the positively charged molecules are configured to amplify the color change effectuated by the nanoparticles. In particular, the positively charged molecules are configured to couple to immobilized nanoparticles (e.g., at the first testing region 106a, second testing region 106b, third testing region 106c, and/or control region 108) to amplify the color change. It is contemplated that the addition of positively charged molecules might increase the color intensity because of their inherent interaction with the negatively charged nanoparticles (e.g., streptavidin capped plasmonic nanoparticles).

The control region 108 may comprise fourth capture compounds, such as fourth capture antibodies. The fourth capture compounds may be immobilized at the control region 108 such that the fourth capture compounds may not flow with the aqueous mixture as it flows through the testing strip 102. The fourth capture compounds are configured to capture the nanoparticles flowing through the testing strip 102. As described above, in embodiments wherein the aqueous mixture has target gene sequences and capture and detection probes coupled to the target gene sequences, at least some of the nanoparticles may nevertheless not bind to the capture and detection probes and may flow with the aqueous mixture as it flows through the testing strip 102 past the testing regions 106a, 106b, and/or 106c. These remaining nanoparticles may be captured by the fourth capture compounds and immobilized at the control region 108. Similarly, in embodiments wherein the aqueous mixture does not have any target gene sequences, the fourth capture compounds may flow freely with the aqueous mixture as it flows through the testing strip 102 and may be captured by the fourth capture compounds and immobilized at the control region 108.

It is contemplated that the fourth capture compounds may be the first small molecules.

As the nanoparticles may be configured to effectuate a color change when reacted and immobilized at a region, when the nanoparticles are immobilized at the control region 108, the nanoparticles may effectuate a color change at the control region 108. In some embodiments, the color change may appear as a visible line or mark at the control region 108. It is contemplated that all proper tests and samples should result in a line or mark at the control region 108, such that the control region 108 ensures the system 100 is working properly.

As seen in FIGS. 1-6, a negative test (e.g., a sample with no respiratory diseases of interest) may result in a line or mark only at the control region 108. As seen in FIG. 1, the control region 108 may be designated by a “C.” A test positive for a first respiratory disease of interest but negative for other respiratory disease(s) of interest may result in a line or mark at the first testing region 106a and a line of mark at the control region 108. As seen in FIG. 1, the first testing region may be designated by a “T1.” A test positive for a second respiratory disease of interest but negative for other respiratory disease(s) of interest may result in a line or mark at the second testing region 106b and a line of mark at the control region 108. As seen in FIG. 1, the second testing region may be designated by a “T2.” A test positive for a third respiratory disease of interest but negative for other respiratory disease(s) of interest may result in a line or mark at the third testing region 106c and a line of mark at the control region 108. As seen in FIG. 1, the third testing region may be designated by a “T3.” A test positive for a first respiratory disease of interest and for a second respiratory disease of interest may result in a line or mark at the first testing region 106a, a line or mark at the second testing region 106b, and a line of mark at the control region 108. A test positive for a first respiratory disease of interest, a second respiratory disease of interest, and a third respiratory disease of interest may result in a line or mark at the first testing region 106a, a line or mark at the second testing region 106b, a line or mark at the third testing region 106c, and a line of mark at the control region 108.

As can be appreciated by the above, the lateral flow system 100 may be configured to detect the presence of one or more respiratory diseases in a collected sample. The lateral flow system 100 may therefore serve as a one step, simultaneous detection method for various respiratory diseases in a POC setting.

As there is an ongoing and immediate need to develop approaches that are low-cost, rapid, do not require the use of advanced equipment, and can be used as a screening tool for the diagnosis of respiratory diseases at POC, it is contemplated that embodiments described herein may provide one or more advantages over currently available screening and detecting techniques. For example, embodiments described herein: (i) do not need prior RNA extraction; (ii) do not demand the use of advanced equipment (e.g., centrifuge, thermocycler, etc.); (iii) do not use conventional pH sensitive dyes; and/or (iv) has a short turnaround time. In some embodiments, the presently described system provides for rapid turnaround time for detection of one or more respiratory diseases. The detection of a respiratory disease of interest may be performed within about 5, 10, 15, 20, 25, or 30 minutes. In one embodiment, detection may be completed within about 15 minutes.

### Respiratory Diseases and Design of ASOs

Embodiments may be configured to detect one or more respiratory diseases, including but not limited to, SARS-COV-2, Flu A, and Flu B. In particular, embodiments of the lateral flow assay may utilize ASOs to detect the presence one or more respiratory diseases present in a sample. The ASOs are designed specifically to bind in complementary fashion to a target gene sequence of a respiratory disease of interest.

To detect SARS-COV-2, at least one target gene sequence correlating to SARS-COV-2 must first be identified such that ASOs can be designed to complement and bind to the sequence. In some embodiments, a target gene sequence correlating to SARS-COV-2 may be chosen from SEQ ID NO 1 (ACACCAAAAGATCACATTGG) and SEQ ID NO 2(CCCGCAATCCTGCTAACAAT). It is contemplated that SEQ ID NO 1 and SEQ ID NO 2 represent two closely spaced apart regions of a target sequence. It is contemplated that these sequences may correlate to nucleocapsid phosphoprotein (N-gene) for SARS-COV-2.

ASOs may then be designed to bind in complementary fashion to target gene sequences of SARS-COV-2. In some embodiments, ASOs may have sequences chosen from a sequence complementary to and configured to bind to SEQ ID NO 1, and a sequence complementary to and configured to bind to SEQ ID NO 2. The ASOs may preferably be designed to target the N-gene sequence of SARS-COV-2 as the analytical sensitivity for N-gene (8.3 copies per reaction) was found to be relatively low compared to RdRP and E genes (3.6 and 3.9 copies per reaction) of SARS-COV-2.

To detect Flu A, at least one target gene sequence correlating to Flu A must first be identified such that ASOs can be designed to complement and bind to the sequence. In some embodiments, a target gene sequence correlating to Flu A may be chosen from SEQ ID NO 3 (CTAGTACTGTGTCTACAGTGTC), SEQ ID NO 4 (ACAGGAAGCAAAGCACAGGG), SEQ ID NO 5 (TCTACAGTGTCAA), and SEQ ID NO 6 (CTAGTACTGTG). It is contemplated that SEQ ID NO 3 and SEQ ID NO 4 represent two closely spaced apart regions of a target sequence, and that SEQ ID NO 5 and SEQ ID NO 6 represent two closely spaced apart regions of a target sequence.

ASOs may then be designed to bind in complementary fashion to target gene sequences of Flu A. In some embodiments, ASOs may have sequences chosen from a sequence complementary to and configured to bind to SEQ ID NO 3, a sequence complementary to and configured to bind to SEQ ID NO 4, a sequence complementary to and configured to bind to SEQ ID NO 5, and a sequence complementary to and configured to bind to SEQ ID NO 6.

To detect Flu B, at least one target gene sequence correlating to Flu B must first be identified such that ASOs can be designed to complement and bind to the sequence. In some embodiments, a target gene sequence correlating to Flu B may be chosen from SEQ ID NO 7 (CGGTGGATTAAACAAAAGC) and SEQ ID NO 8 (GCCAATGGAACCAAATATAG). It is contemplated that SEQ ID NO 7 and SEQ ID NO 8 represent two closely spaced apart regions of a target sequence.

ASOs may then be designed to bind in complementary fashion to target gene sequences of Flu B. In some embodiments, ASOs may have sequences chosen from a sequence complementary to and configured to bind to SEQ ID NO 7, and a sequence complementary to and configured to bind to SEQ ID NO 8.

Although sequence listings are provided for SARS-COV-2, Flu A, and Flu B, it is contemplated that the methods and systems described herein may be configured to screen and detect any respiratory disease of interest.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The ASOs were analyzed according to their binding affinity and disruption energy to the target gene sequences. The ASOs has optimum GC content with better binding and disruption energy. The N-gene, i.e., nucleocapsid phosphoprotein, of SARS-COV-2 was chosen and ASOs were selected to target two closely following region (see FIG. 2). These ASOs, ASO1 and ASO2, were labeled with biotin and FAM at their 5′ and 3′ end respectively. These ASOs were subsequently used for sensing purposes using a lateral flow strip. Briefly, 3′-amine terminated six carbon spaced ASO2 (ASO2-3′-C6-NH2, 25 μM) was mixed with FAM (30 μM) overnight at room temperature in dark to get FAM labeled ASO2. Also, 5′-amine terminated six carbon spaced ASO1 (NH2-C6-5′-ASO1, 25 μM) was mixed EDC/NHS/DMAP (37.5/37.5/0.25 μM) along with biotin (30 μM) overnight at room temperature to get biotin labeled ASO1. Both the ASOs after conjugation were purified by sephadex G-25 column using RNase free water as eluent to remove unbounded FAM and biotin. The ASOs were designed in such a way that they can selectively detect two regions of the N-gene of SARS-COV-2 which will ensure that the test will not fail even if one region of the viral gene undergoes mutation during its current spread. In a similar manner, ASO3 and ASO4 conjugated at their 5′ and 3′ end with DIG and FAM respectively were used for the detection of pangenotypic Flu A/B.

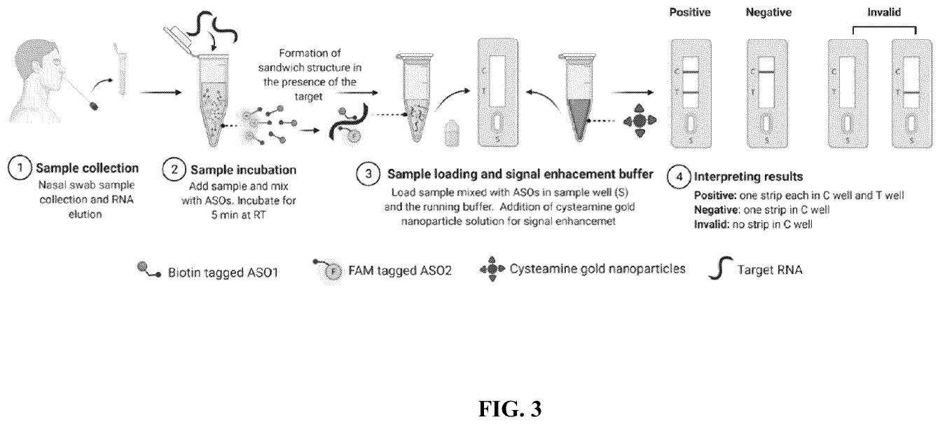

Regarding the purification technique mentioned above, an RNA extraction-free technique that utilizes a Sephadex G25 DNA purification size exclusion column (NAP-10) for rapid RNA isolation was used. Briefly, NAP-10 column was equilibrated as per the manufacturer's protocol. 40 μL of the nasopharyngeal swab sample in viral transport media (VTM) was mixed with 20 μL of guanidine isothiocyanate containing lysis buffer and added to the NAP-10 column. 1 ml of RNase free water was added to the column and the eluted liquid containing the RNA was collected. Separately, we labeled the previously identified ASOs with FAM and biotin respectively. The isolated viral RNA was then added to the FAM and biotin labeled ASOs and allowed to incubate for 5 minutes. The resulting mixture (20 μL) along with running buffer (85 μL) was then added to a lateral flow strip and the result is read with naked eyes after 5 mins. As the products migrate along the strip, the FAM/biotin labeled ASOs bind with their target SARS-CoV-2 viral RNA. This assembly is then captured by the anti-FAM antibody immobilized at the test line (T) because of the presence of FAM-labeled ASOs. The streptavidin coated gold nanoparticles are then attracted by the biotin labeled ASOs leading to the formation of a faint red T line. Now to enhance the visibility and increase the contrast of the test line (T), we have used cysteamine capped gold nanoparticles (cyst-AuNPs) for signal augmentation. In this case, the positively charged cysteamine capped gold nanoparticles bind strongly with the negatively charged streptavidin coated gold nanoparticles leading to a prominent visible band on the test line indicating the presence of SARS-COV-2 as shown in FIG. 2. FIG. 3 describes the workflow of the developed assay.

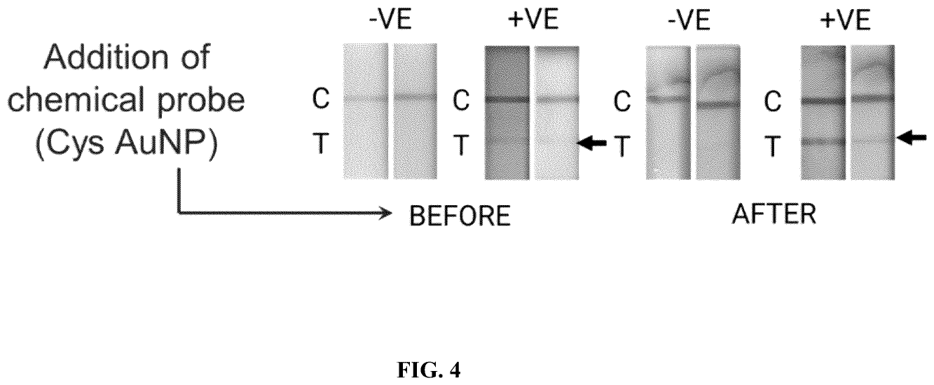

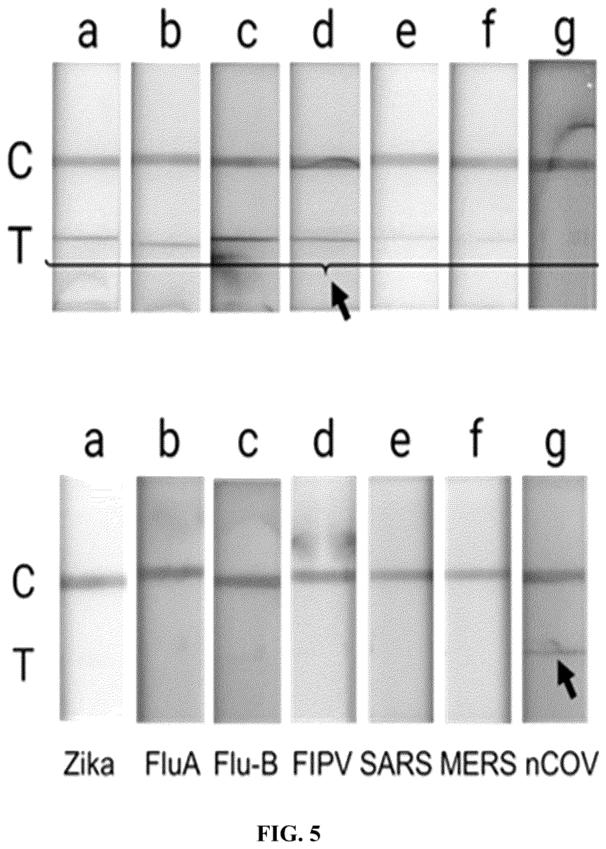

FIG. 4 shows the results from a study where four COVID-19 clinical samples (2 positive and 2 negative samples) have been tested using our strategy. While no bands are visible in case of the negative samples the test bands are visible in case of the positive samples. The rest of the excess unreacted streptavidin coated particles keep moving and are captured by biotin immobilized at the control line (C). This step can help verify the correct operation of the lateral flow system. It only takes 5 min to allow the completion of the operating procedure from sample loading to signal readout with the LFA. Additionally, the signal amplified bands with the addition of cyst-AuNPs are shown in FIG. 4 which further strengthens our sensing approach. A faint test line (T) is observed before addition of cysteamine AuNPs in case of positive samples which becomes more prominent after the addition of cysteamine AuNPs indicating the presence of SAR-COV-2. On the other hand, the test line is not visible in both cases (before and after addition of cys-AuNPs) when the lateral flow strip is tested with negative samples. Additionally, FIG. 5 showed the results from our preliminary sensitivity and selectivity study where we utilized SARS-COV-2 genomic RNA to investigate the analytic detection limit of the LFA system for SARS-COV-2. Briefly, 10-fold serial dilutions of the SARS-COV-2 genomic RNA with concentrations ranging from 67250 copies/μL to 0.001 copies/μL were used to test the developed LFA system (FIG. 5).

We confirmed that the assay can generate a clear visible band up to 8 copies/μL of SARS-COV-2 RNA and a relatively faint visible band when the concentrations of SARS-COV-2 genomic RNA are ≥0.02 copies/μL. Moreover, we further tested the LFA system's specificity for SARS-COV-2 diagnostics. SARS-COV-2 genomic RNA and 6 types of synthetic plasmids/viral RNA from other common pathogenic microorganisms (FIG. 5), were tested on the RT-LAMP-LFA platform, and no positive signals (red T line) were observed except for SARS-COV-2. The results showed that there is no cross-reaction of this designed system with other pathogenic microorganisms.

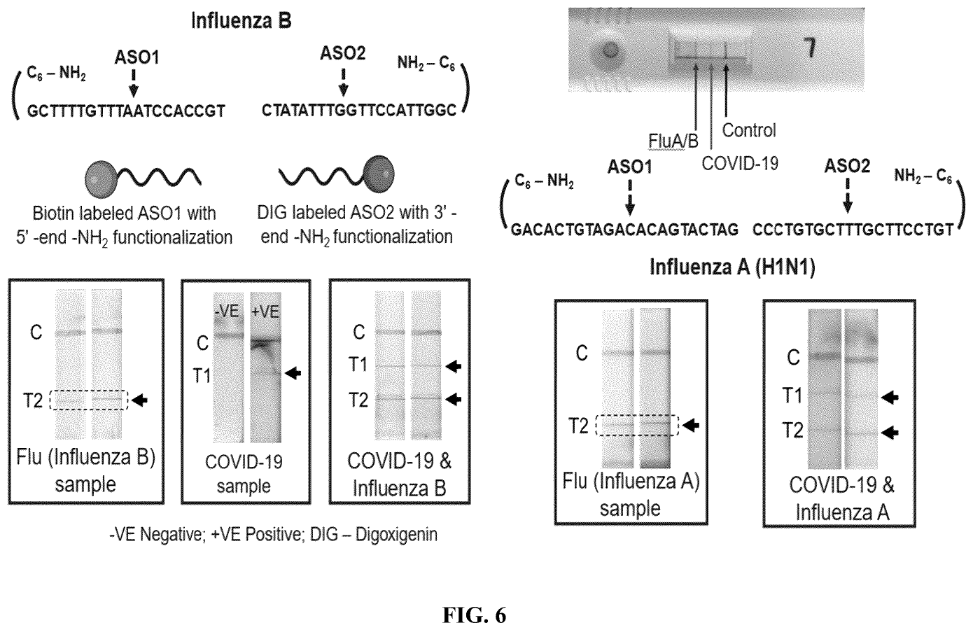

To demonstrate multiplex detection, we designed the ASO's towards the hemagglutinin gene of pangenotypic Flu A/B. Subsequently, we labeled the identified ASO's with biotin and digoxigenin (DIG) respectively and tested them against Flu A and B using the lateral flow strip. Here 5′ end of ASO3 (sequence: GACACTGTAGACACAGTACTAG) has been tagged with DIG and 3′ end of ASO4 (sequence: CCCTGTGCTTTGCTTCCTGT) has been tagged with 6-FAM to selectively diagnose pangenotypic Flu A/B. FIG. 6 showed the results from the preliminary study. The complete lateral flow strip tested against SARS-COV-2 and Flu B can be seen in FIG. 6 showed the preliminary results when the lateral flow strip was tested against SARS-COV-2, Influenza A/B.

The selected ASOs should bind to SARS-COV-2 and Flu RNA with high specificity and negligible off-target binding. The successful synthesis of nanoparticles will be confirmed by recording the absorbance and follow the plasmonic peak at ˜520 nm. The synthesized positively charged cysteamine-coated nanoparticles should enhance the signal indicating the presence of the viral RNA.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and process and/or utilization and methods of making and using the same have been discussed and illustrated herein, it is to be distinctly understood that the invention is not limited thereto but may be otherwise variously embodied and practiced within the scope of the following claims.

What is claimed is:

1. An apparatus for detecting one or more respiratory diseases in a sample, the apparatus comprising:
first antisense oligonucleotides functionalized with a first small molecule at their first ends, wherein the first antisense oligonucleotides have a sequence that is complementary of a first target gene sequence of a first respiratory disease;
second antisense oligonucleotides functionalized with a second small molecule at their second ends, wherein the second antisense oligonucleotides have a sequence that is complementary of a second target gene sequence of the first respiratory disease;
third antisense oligonucleotides functionalized with a third small molecule at their first ends, wherein the third small molecule is the same as the first small molecule, wherein the third antisense oligonucleotides have a sequence that is complementary of a first target gene sequence of a second respiratory disease; and
fourth antisense oligonucleotides functionalized with a fourth small molecule at their second ends, wherein the fourth small molecule is different than the second small molecule, wherein the fourth antisense oligonucleotides have a sequence that is complementary of a second target gene sequence of the second respiratory disease.

2. The apparatus of claim 1, wherein the first and second respiratory diseases are two different diseases selected from the group consisting of SARS-COV-2, Flu A, and Flu B.

3. The apparatus of claim 1, further comprising:
fifth antisense oligonucleotides functionalized with a fifth small molecule at their first ends, wherein the fifth small molecule is the same as the first and third small molecules, wherein the fifth antisense oligonucleotides have a sequence that is complementary of a first target gene sequence of a third respiratory disease; and
sixth antisense oligonucleotides functionalized with a sixth small molecule at their second ends, wherein the sixth small molecule is different than the second and fourth small molecules, wherein the sixth antisense oligonucleotides have a sequence that is complementary of a second target gene sequence of the third respiratory disease.

4. The apparatus of claim 3, wherein the first, second, and third respiratory diseases are three different diseases selected from the group consisting of SARS-COV-2, Flu A, and Flu B.

5. The apparatus of claim 1, wherein when the first or second respiratory disease is SARS-CoV-2, the first and second target gene sequences of SARS-COV-2 are ACACCAAAAGATCACATTGG and CCCGCAATCCTGCTAACAAT,
wherein when the first or second respiratory disease is Flu A, the first and second target gene sequences of Flu A are selected from:
CTAGTACTGTGTCTACAGTGTC and ACAGGAAGCAAAGCACAGGG, and
TCTACAGTGTCAA and CTAGTACTGTG, and
wherein when the first or second respiratory disease is Flu B, the first and second target gene sequences of Flu B are CGGTGGATTAAACAAAAGC and GCCAATGGAACCAAATATAG.

6. The apparatus of claim 1, wherein the first small molecule and the third small molecule is a small molecule selected from the group consisting of biotin, 6-carboxyfluorescein (6-FAM), fluorescein isothiocyanate (FITC), and digoxigenin (DIG).

7. The apparatus of claim 1, wherein the second small molecule and fourth small molecule are two different small molecules selected from the group consisting of biotin, 6-carboxyfluorescein (6-FAM), fluorescein isothiocyanate (FITC), and digoxigenin (DIG).

8. The apparatus of claim 1, further comprising:
a testing strip comprising a sample application region, a control region, a first testing region configured to detect the presence of the first respiratory disease, and a second testing region configured to detect the presence of the second respiratory disease, wherein the first and second testing regions are positioned between the sample application region and the control region, and
wherein the sample application region is configured to receive the sample, which is configured flow through the testing strip towards the first and second testing regions and the control region.

9. The apparatus of claim 8, wherein the first testing region has first capture compounds immobilized on the testing strip, wherein the first capture compounds are configured to capture the second small molecule of the second antisense oligonucleotides.

10. The apparatus of claim 9, wherein the second testing region has second capture compounds immobilized on the testing strip, wherein the second capture compounds are configured to capture the fourth small molecule of the fourth antisense oligonucleotides.

11. The apparatus of claim 10, wherein the testing strip has nanoparticles configured to flow through the testing strip, wherein some of the nanoparticles are configured to capture the first small molecule of the second antisense oligonucleotides, and wherein some of the nanoparticles are configured to capture the third small molecule of the fourth antisense oligonucleotides.

12. The apparatus of claim 11, wherein the nanoparticles are configured to effectuate a color change when reacted and immobilized on the testing strip.

13. The apparatus of claim 12, further comprising positively charged molecules configured to flow through the testing strip, wherein the positively charged molecules are configured to bind to the nanoparticles and augment the color change effectuated by the nanoparticles.

14. The apparatus of claim 8, wherein the control region has third capture compounds immobilized on the testing strip, wherein the third capture compounds are configured to capture some of the nanoparticles.

15. A method for detecting one or more respiratory diseases in a sample, the method comprising:
providing a sample solution comprising:
a collected sample including nucleic acid from a subject,
first antisense oligonucleotides functionalized with a first small molecule at their first ends, wherein the first antisense oligonucleotides have a sequence that is complementary of a first target gene sequence of a first respiratory disease,
second antisense oligonucleotides functionalized with a second small molecule at their second ends, wherein the second antisense oligonucleotides have a sequence that is complementary of a second target gene sequence of the first respiratory disease,
third antisense oligonucleotides functionalized with a third small molecule at their first ends, wherein the third small molecule is the same as the first small molecule, wherein the third antisense oligonucleotides have a sequence that is complementary of a first target gene sequence of a second respiratory disease, and
fourth antisense oligonucleotides functionalized with a fourth small molecule at their second ends, wherein the fourth small molecule is different than the second small molecule, wherein the fourth antisense oligonucleotides have a sequence that is complementary of a second target gene sequence of the second respiratory disease;
providing a testing strip comprising a sample application region, a control region, a first testing region configured to detect the presence of the first respiratory disease, and a second testing region configured to detect the presence of the second respiratory disease, wherein the first and second testing regions are positioned between the sample application region and the control region; and
applying the sample solution at the sample application region.

16. The method of claim 15, wherein the first and second respiratory diseases are two different diseases selected from the group consisting of SARS-COV-2, Flu A, and Flu B.

17. The method of claim 15, the sample solution further comprising:
fifth antisense oligonucleotides functionalized with a fifth small molecule at their first ends, wherein the fifth small molecule is the same as the first and third small molecules, wherein the fifth antisense oligonucleotides have a sequence that is complementary of a first target gene sequence of a third respiratory disease; and
sixth antisense oligonucleotides functionalized with a sixth small molecule at their second ends, wherein the sixth small molecule is different than the second and fourth small molecules, wherein the sixth antisense oligonucleotides have a sequence that is complementary of a second target gene sequence of the third respiratory disease.

18. The method of claim 17, wherein the first, second, and third respiratory diseases are three different diseases selected from the group consisting of SARS-COV-2, Flu A, and Flu B.

19. The method of claim 15, wherein when the first or second respiratory disease is SARS-CoV-2, the first and second target gene sequences of SARS-COV-2 are ACACCAAAAGATCACATTGG and CCCGCAATCCTGCTAACAAT,
wherein when the first or second respiratory disease is Flu A, the first and second target gene sequences of Flu A are selected from:
CTAGTACTGTGTCTACAGTGTC and ACAGGAAGCAAAGCACAGGG, and
TCTACAGTGTCAA and CTAGTACTGTG, and
wherein when the first or second respiratory disease is Flu B, the first and second target gene sequences of Flu B are CGGTGGATTAAACAAAAGC and GCCAATGGAACCAAATATAG.

20. The method of claim 15, wherein the first testing region has first capture compounds immobilized on the testing strip, wherein the first capture compounds are configured to capture the second small molecule of the second antisense oligonucleotides.

21. The method of claim 20, wherein the second testing region has second capture compounds immobilized on the testing strip, wherein the second capture compounds are configured to capture the fourth small molecule of the fourth antisense oligonucleotides.

22. The method of claim 21, wherein the testing strip has nanoparticles configured to flow through the testing strip, wherein some of the nanoparticles are configured to capture the first small molecule of the firth antisense oligonucleotides, and wherein some of the nanoparticles are configured to capture the third small molecule of the third antisense oligonucleotides.

23. The method of claim 22, wherein the nanoparticles are configured to effectuate a color change when reacted and immobilized on the testing strip.

24. The method of claim 23, further comprising applying positively charged molecules configured to flow through the testing strip, wherein the positively charged molecules are configured to bind to the nanoparticles and augment the color change effectuated by the nanoparticles.
